(19) United States
(12) Patent Application Publication
(10) Pub. No.: US 2006/0253000 A1
(43) Pub. Date: Nov. 9, 2006

(54) Method of temporarily viewing a physiological parameter of another

(76) Inventor: Richard D. Ciervo, New Britain, CT (US)

Correspondence Address: Arthur G. Schaier;Carmody & Torrance LLP, 50 Leavenworth Street, P.O. Box 1110, Waterbury, CT, 06721-1110, US

(21) Appl. No.: 11/122,619

(22) Filed: May 5, 2005

(51) Int. Cl. A61B 5/00; A61B 5/04

(52) U.S. Cl. 600/300; 128/903; 600/509

(57) ABSTRACT

A method of displaying, on a display of an electronic device, a physiological parameter of at least a first individual having associated therewith a first biomedical sensing device and a second individual having associated therewith a second biomedical sensing device, the method comprising the steps of, while maintaining a wireless communication link between the first biomedical sensing device and the electronic device, initiating a wireless communication link between the second biomedical sensing device and the electronic device such that information packets related to the physiological parameter of the second individual are transmitted from the second biomedical sensing device and received and processed by the electronic device; and displaying, on the display, the physiological parameter of the second individual based on the information packets received and processed by the electronic device. An arrangement that carries out the foregoing method is also contemplated hereby.

### BACKGROUND OF THE INVENTION

The present invention is directed to physiological parameter monitors in general, and in particular, to a novel arrangement and methodology for displaying, on a display of an electronic device, a physiological parameter of at least a first individual having associated therewith a first biomedical sensing device and a second individual having associated therewith a second biomedical sensing device. The biomedical sensing devices detect the physiological parameters of the respective individuals. While not limited thereto, the present invention can be well appreciated and can be utilized in exercise, coaching, supervising, athletic and/or teaching endeavors, just to name a few.

A review of the prior art will reveal a plurality of embodiments related to devices for measuring physiological parameters, such as heart rate, blood pressure and oxygen levels, just to name a few.

By way of example, one of the most popular heart rate monitoring arrangements comprise a transmitter device that is preferably (but not necessarily) worn about one's chest and a corresponding receiver, which is also worn about the user, preferably about the wrist. Timex Corporation is but one leading manufacturer of such arrangements.

One of the perceived concerns when designing such arrangements is the undesirable possibility of one receiver in a transmitter/receiver pair picking up and processing (e.g. and even possibly displaying) the transmitted signal of another transmitter. That is, it is a perceived disadvantage if two side-by-side runners cannot be assured that their respective displays are displaying their own heart rate, and not that of the other. More specifically, in those most popular heart rate monitor arrangements using wireless data transmission from the sensor/transmitter (e.g. chest) unit to the display (e.g. wrist worn) unit, it is important to reduce or eliminate interference that may occur when a person wearing a heart monitor is in close proximity to another person wearing another heart monitor.

However, there may be instances when it would be desirable to provide an arrangement whereby a teacher, coach, supervisor, teammate or the like could temporarily (or continuously if desired) obtain the physiological parameter information from an individual other than him/herself. Such an arrangement is counterintuitive from the arrangements currently found in the art which strive and strain to ensure the elimination of processing of nearby or proximate transmitters. For example, in a one-on-one training or educational environment, it would be advantageous if one individual, while monitoring his/her own physiological parameter(s), could temporarily (or continuously if desired) view the physiological parameter of the other person.

Similarly, in a group activity, it would be desirable if there were an arrangement and/or methodology to permit the teacher, coach, supervisor or teammate to temporarily (or continuously if desired) view the measured physiological parameter of that of another selected person from the group. As but a specific example, it would be advantageous if a health instructor, while monitoring her own heart rate, could temporarily (or continuously if desired) view a measurable physiological parameter of one or more of the other class participants.

The present invention overcomes the perceived deficiencies in the prior art and provides the advantages disclosed herein.

### SUMMARY AND OBJECTIVES OF THE INVENTION

It is thus an objective of the present invention to overcome perceived deficiencies in the prior art, such as the present state of the art's inability to provide an arrangement or method to carry out the foregoing functionality.

In particular, it is an objective of the present invention to provide an arrangement and methodology for displaying, on a display of an electronic device, one or more physiological parameters of at least two individuals in a desirable, accurate and informative manner.

It is another objective of the present invention to provide an arrangement and methodology to selectively view the measurable physiological parameter(s) of another.

It is yet another objective of the present invention to provide the foregoing arrangement and methodology in an electronic device/biomedical sensing device combination in which signals from the biomedical sensing devices are transmitted wirelessly to the electronic devices.

Further objects and advantages of this invention will become more apparent from a consideration of the drawings and ensuing description.

The invention accordingly comprises the features of construction, combination of elements, arrangement of parts and sequence of steps which will be exemplified in the construction, illustration and description hereinafter set forth, and the scope of the invention will be indicated in the claims.

Generally speaking, the present invention is directed to a method of displaying, on a display of an electronic device, a physiological parameter of at least a first individual having associated therewith a first biomedical sensing device and a second individual having associated therewith a second biomedical sensing device, the method comprising the steps of: initiating a communication link between the first biomedical sensing device and the electronic device such that information packets related to the physiological parameter of the first individual are transmitted from the first biomedical sensing device and received and processed by the electronic device; displaying, on the display, the physiological parameter of the first individual based on the information packets received and processed by the electronic device; and while maintaining a wireless communication link between the first biomedical sensing device and the electronic device, the method further comprises the steps of: initiating a wireless communication link between the second biomedical sensing device and the electronic device such that information packets related to the physiological parameter of the second individual are transmitted from the second biomedical sensing device and received and processed by the electronic device; displaying, on the display, the physiological parameter of the second individual based on the information packets received and processed by the electronic device.

An arrangement that carries out the foregoing method is also contemplated hereby.

### BRIEF DESCRIPTION OF THE DRAWINGS

For a fuller understanding of the invention, reference is had to the following description taken in connection with the accompanying figures, in which:

FIG. 1 is a illustration of a plurality of individuals each associated with an electronic device and a biomedical sensing device in accordance with the present invention and in an environment contemplated thereby;

FIG. 2 is a block diagram of a biomedical sensing device utilized in the present invention;

FIGS. 3A and 3B are each timing diagrams illustrating heart rate information being transmitted by two respective biomedical sensing devices, and FIG. 3C illustrates the display of heart rate information from the two biomedical sensing devices by an electronic device constructed in accordance with the present invention, it being understood that all of the diagrams in FIG. 3 are deemed to be “timewise” aligned;

FIG. 4 is flowchart of a sequence of steps in accordance with the present invention; and

FIG. 5 is a block diagram of an electronic device constructed in accordance with the present invention.

Identical reference numerals in the figures are intended to indicate like features, although not every feature in every figure may be called out with a reference numeral.

### DETAILED DESCRIPTION OF THE PREFERRED EMBODIMENTS

Generally speaking, the present invention provides a method of displaying, on a display of an electronic device, a physiological parameter of at least a first individual having associated therewith a first biomedical sensing device and a second individual having associated therewith a second biomedical sensing device. An environment in which the present invention is applicable may be, by way of example and not limitation, a health club, an educational environment, an athletic environment, just to name a few.

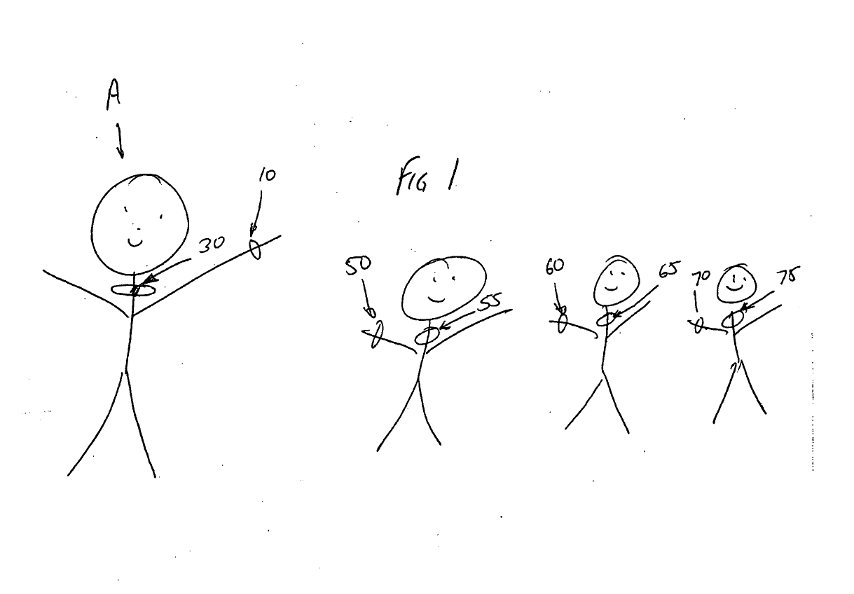

FIG. 1 is intended to generically illustrate an applicable environment, and in particular, illustrates a first individual A, who could (but need not) be a teacher, coach, supervisor or the like and one or more additional individuals B, C and D, each of which could be students, participants in an activities, etc.

In accordance with the invention, at least individual A has associated therewith an electronic device, generally indicated at 10, constructed in accordance with the present invention. In the preferred embodiment, electronic device 10 may be worn about one's wrist. Associated with individual A is also a first biomedical sensing device, generally indicated at 30, for sensing a physiological parameter of individual A. As illustrated in FIG. 1, the positioning of biomedical sensing device 30 about the chest of individual A makes it most applicable as a heart rate monitor, but this is by way of example and not limitation. That is, it should be clear that the physiological parameter to which the present invention is directed may be something other than heart rate, such as blood pressure, oxygen levels in the blood, just to name a few.

Also illustrated in FIG. 1 is a plurality of other individuals B, C and D, each of which also has associated therewith an electronic device constructed in accordance with the prior art or the present invention and generally indicated at 50, 60 and 70, respectively. While practically speaking, all four electronic devices 10, 50, 60 and 70 may be constructed identically, it will become clear that the patentable features of the electronic device of the present invention need not be included in each individual's electronic device. In fact, it is possible that the electronic devices of those other than individual A may be constructed in accordance with the state of the art and as further disclosed below.

FIG. 1 also illustrates each of the individuals A, B, C and D as being associated with a biomedical sensing device. Again, as the primary patentable features of the present invention lie primarily in the electronic device, it is possible that the biomedical sensing devices associated with each individual are all constructed in accordance with the present state of the art. For completeness, individual A is associated with biomedical sensing device 30, and individuals B, C and D are each associated with a biomedical sensing device generally indicated at 55, 65 and 75, respectively. Again, at the risk of being redundant, in the example of FIG. 1, each of the biomedical sensing devices are heart rate monitors. However, other biomedical sensing devices, such as blood pressure, oxygen levels and others are contemplated herein, and may be generically seen to be described in FIG. 1.

As would be understood by one ordinarily skilled in the art, there are at least several methodologies for transmitting the sensed data relating to the physiological parameter from the respective sensing devices (e.g. 30, 55, 65, 75) to its associated electronic device (e.g. 10, 50, 60, 70). For example, in one preferred embodiment, the biomedical sensing device transmits a packet of data (comprising among other things, the heart rate itself) in regular intervals (e.g. every two (2) seconds). With this transmission methodology, the transmission rate is independent of the actual heart rate (to be contrasted with a methodology to be described below) that the respective sensing device is measuring. In the event there is no identification scheme to permit an electronic device to differentiate between the packets of information being transmitted by more than one transmitter, a preferred protocol is for the electronic device to first “acquire” a first valid information packet, and then configure itself to know that every regular interval thereafter (e.g. the aforementioned two (2) seconds), another information packet will be transmitted (and presumably from the sensing device from which the electronic device “acquired” the first valid information packet).

The protocol in connection with this first methodology comprises the utilization of an acquisition mode and a synchronous mode. In the acquisition mode, the electronic device powers up and waits for a valid information packet (based on an expected bit length, etc.). As soon as the electronic device receives the information packet, it's time of reception (e.g. relative to an internal timer) is recorded and the electronic device enters its synchronous mode. In the synchronous mode, the receiver in the electronic device can then be “shut off” essentially until it expects to receive the next information packet (e.g. two (2) seconds later). At that point, the receiver in the electronic device preferably turns on just in time to catch the next packet from (presumably) the same transmitter. This process is then repeated every interval of time (e.g. every two (2) seconds) to obtain the next information packet containing the heart rate sensed and computed in the biomedical sensing device.

As one skilled in the art would understand, the foregoing methodology comprises among other things, the steps of obtaining a signal of heartbeat pulses from the individual and determining the heart rate of the individual from the detected (e.g. ECG) signal. Preferably, the heart rate is determined in the biomedical sensing device.

The foregoing method can easily be contrasted with a methodology in which the rate of transmission of the information packets may be used to determine the heart rate. Such a methodology is described in U.S. Pat. No. 5,538,007, the subject matter of which is incorporated by reference as if fully set forth herein. This '007 patent describes a methodology in which the biomedical sensing device produces an encoded digital signal having a transmission rate indicative of the heart rate. Additionally, the bit sequence of the transmitted packet includes an identification of the particular biomedical sensing device from which the transmission was made. Moreover, as best understood, the method described in this '007 patent preferably computes the heart rate in the electronic device itself.

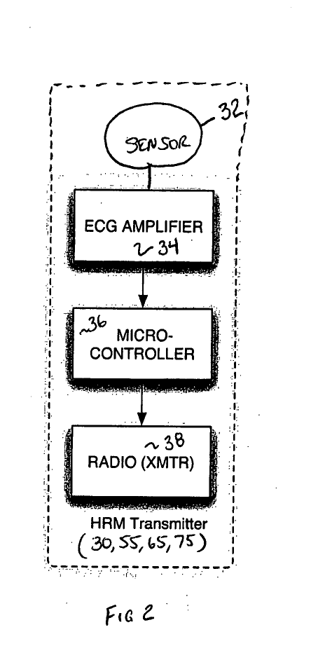

FIG. 2 illustrates an exemplary biomedical sensing device, which represents any and/or all of the biomedical sensing devices illustrated in FIG. 1, namely sensing devices 30, 55, 65 and 75. More generally, FIG. 2 also illustrates a biomedical sensing device for sensing and transmitting information packets regarding other physiological parameters, such as blood pressure and/or oxygen levels.

Specifically illustrated in FIG. 2, the biomedical sensing device comprises a physiological parameter sensor 32 which is coupled to an amplifier 34, which when detecting heartbeats, is preferably an ECG amplifier, which itself is operatively coupled to a microcontroller 36 for processing the amplified ECG signal. The signal, once processed and properly formatted in accordance with the aforementioned protocols, is then transmitted by a transmitter 38, which itself is coupled to microcontroller 36.

In accordance with the prior art and as implied above, it has been previously preferable that only the related electronic device associated with each individual processes the transmitted information packets from the respective sensing device. That is, it has been preferable that electronic device 10 only processes the information from and displays the sensed parameter of the individual associated with biomedical sensing device 30.

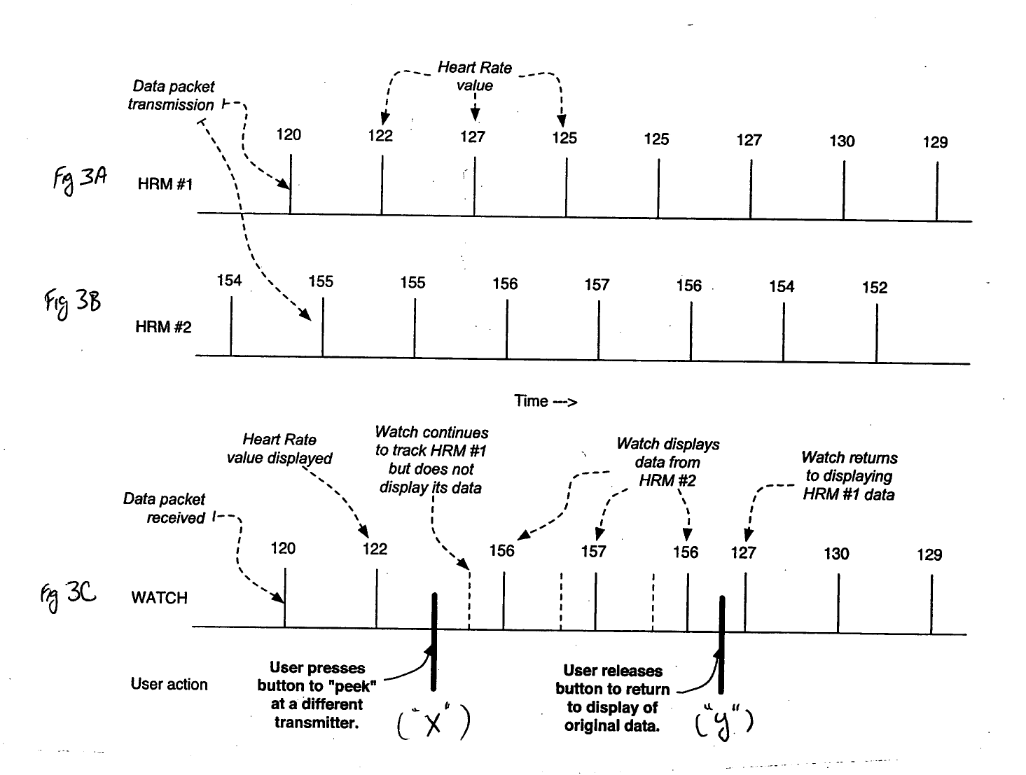

Reference is next made to FIGS. 3A, 3B and 3C, which illustrates a timing chart in accordance with the present invention. As described above, an exemplary implementation of the present invention is that in which an instructor or coach may wish to temporarily (or continuously) view the heart rate data from a student (or athlete) on his/her (i.e. the instructor/coach's) own electronic device. Several embodiments are contemplated.

For example, the coach/instructor (e.g. individual A) can simply approach the athlete (e.g. individual B or C or D), push an actuator (e.g. a push-button) and, within a few seconds (as more specifically disclosed below), see the heart rate of individual B on the display of electronic device 10. However, and importantly, and in accordance with the present invention, the communication link between biomedical sensing device 30 and electronic device 10 is maintained during the viewing of the heart rate of individual B on the electronic device of individual A. This feature is very advantageous and patentably different from prior art arrangements that would require a reinitialization of the communication link between sensing device 30 and electronic device 10. By maintaining the original communication link between sensing device 30 and electronic device 10, a much more versatile, advantageous and useful physiological parameter monitoring system is provided.

That is, the ability to intentionally “peek” at the heart rate of an individual (e.g. individual B) on the electronic device of another (e.g. electronic device 10 of individual A) is achievable because of the first mentioned methodology wherein each biomedical sensing device and electronic device pair employ a predictable packetized timing scheme of communication. That is, in accordance with the present invention, the timing sequence of the transmission of information packets between biomedical sensing device 30 and electronic device 10 (e.g. that of individual A) is not “forgotten”.

For example, FIGS. 3A and 3B illustrate exemplary sequences of transmission of information packets from respective biomedical sensing devices (e.g. sensing device 30 and sensing device 55). In accordance with the methodology disclosed above, at every predicted interval (e.g. 2 seconds) the respective biomedical sensing devices are transmitting their respective information packets. As illustrated, the heart rate of individual A is in the range of 120 to 130 beats per minute and the heart rate of individual B is in the range of 152 to 157 beats per minute.

In accordance with the present invention, FIG. 3C illustrates how individual A can temporarily (or continuously) view the heart rate of individual B on his/her own electronic device. Specifically, the illustrated sequence discloses that the communication link between biomedical sensing device 30 and electronic device 10 is established in accordance with the methodology disclosed above, and in this way, electronic device 10 first displays the first two heart rate values (e.g. 120 bpm and 122 bpm) transmitted 2 seconds apart, determined from the information packets transmitted by sensing device 30.

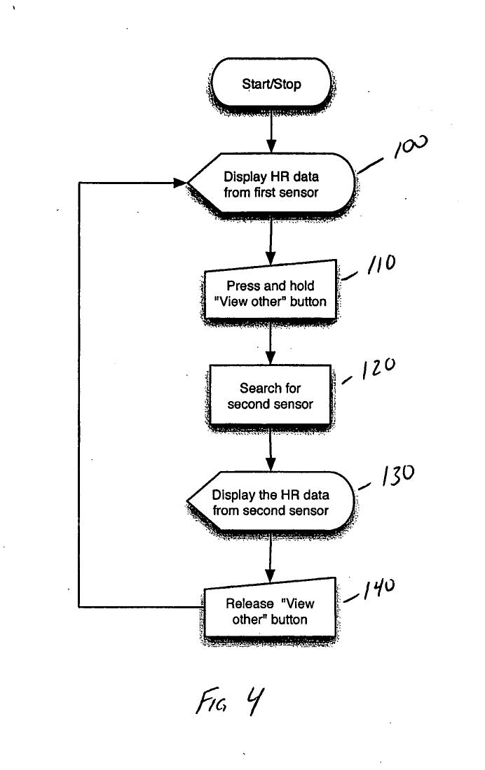

FIG. 3C in combination with FIG. 4 illustrates a synchronization sequence (e.g. via a pusher or the like on electronic device 10) in which electronic device 10 also establishes a communication link with biomedical sensing device 55 in the manner disclosed above, all the while maintaining its communication link with biomedical sensing device 30. Specifically, FIG. 3C illustrates an exemplary methodology in accordance with the present invention in which, while an actuator remains actuated (e.g. individual A maintains compression of a pusher or the like), the display of heart rate information being transmitted from biomedical sensing device 30 is temporarily inhibited from display on device 10 while the display of heart rate information from biomedical sensing device 55 takes place on the display of electronic device 10. At any desired time (e.g. time “Y” and illustrated by example by the release of the pusher), electronic device ceases its display of heart rate information from biomedical sensing device 55 and returns to its display of heart rate information from sensing device 30.

In particular, as illustrated in FIG. 4 at step 100, electronic device 10 is displaying heart rate data from sensing device 30 (i.e. it is assumed that the communication link has already been established as set forth above). At step 110 and at moment “X”, the user actuates a pusher or the like. At step 120, electronic device 10 begins its search for the desired “second” sensing device (i.e. sensing device 55). At step 130, electronic device 10 begins to display the heart rate data being transmitted by sensing device 55 (namely heart rate information corresponding to 156 bpm, 157, bpm, 156 bpm) while also inhibiting the display of the heart rate information being transmitted by sensing device 30. At step 140 (and at time “Y”), a simple release of the actuator causes electronic device 10 to resume its display of the heart rate information being transmitted by sensing device 30. FIG. 3C again illustrates that the present invention does not require any reestablishing of a communication link between electronic device 10 and sensing device 30 since the communication link was maintained during the sequence disclosed in FIG. 4.

The ability for electronic device 10 to maintain its communication link with biomedical sensing device 30 while processing and displaying the sensed physiological parameter data of sensing device 55 provides a significant improvement over state of the art wireless arrangements which would require (i.e. in order to resume the display of heart rate information being transmitted from sensing device 30 on electronic device 10) individual A to resynchronize its communication link, which would be undesirable.

To be sure, the present invention also contemplated the temporary display of the second heart rate information (e.g. that of biomedical sensing device 55) with only one actuation. That is, a single actuation (e.g. button push and release) on electronic device 10 may provide for the temporary display of the physiological parameter (e.g. heart rate) of individual B for a predetermined number of seconds. That is, there is no requirement that there be a maintenance of the button push during the entire displaying of the alternate heart rate information. To this end, a preferred predetermined number of seconds for the temporarily display of a second heart rate is on the order of 6-15 seconds.

Several methods of establishing the communication link between electronic device 10 and the second biomedical sensing device (e.g. device 55) are contemplated herein. It its simplest scheme, electronic device 10 would establish its second communication link with the biomedical sensing device from which it receives the strongest signal. Such a scheme works well in an environment, for example, whereby electronic device 10 is in close proximity with biomedical sensing device 55.

However, a further enhancement provides each biomedical sensing device with not only an address, but an “identification tag” that allows electronic device 10 to select the desired biomedical sensing device with which it will establish a communication link. In this way, for example, individual A can select, from a plurality of individuals (e.g. individuals B, C or D), the biomedical sensing device which electronic device will establish the secondary communication link. The incorporation of an “identification” tag into the transmitted packet sequence is advantageous when there are a plurality of secondary biomedical sensing devices (e.g. devices 55, 65, 75) from which to choose from and it is not desirable or practical to move into close proximity with the desired sensing device. Using an identification tag provides greater versatility when the transmitters of the biomedical sensing devices have a relatively long transmission range.

Although the timing sequence of FIGS. 3A, 3B and 3C illustrate a protocol in which information packets are transmitted in predetermined intervals, the present invention is equally adaptable to use with the one or more transmission protocols described in the aforementioned U.S. Pat. No. 5,538,007.

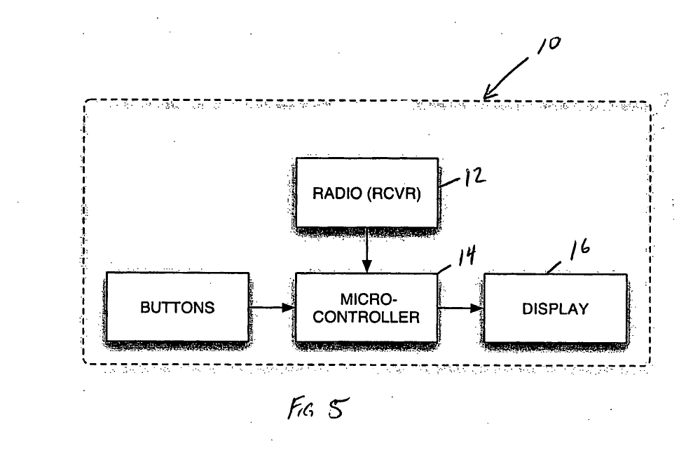

FIG. 5 illustrates a block diagram of electronic device 10, with the electronic devices associated with individuals B, C and D either being identical or constructed in accordance with the present state of the art. Specifically, electronic device 10 preferably comprises a receiver 12 for receiving wireless transmissions from one or more of the sensing devices, a controller 14, operatively coupled to receiver 12, for processing the received signal in accordance with the methodologies set forth above, and a display (e.g. LCD) unit 16 for displaying the physiological parameter sensed by the sensor of the respective sensing device. A set of pushers 18 may be provided to carry out normal display and setting functions, one of which may be used to initiate the temporary display of the physiological parameter information of another as set forth above.

While maintaining the communication link between the first biomedical sensing device and the electronic device, the foregoing method generally comprises the steps of displaying, on the display, the physiological parameter of the second individual based on the information packets received and processed by the electronic device.

However, various display alternatives are provided. For example, in one embodiment, the method comprises the steps of inhibiting the display of the physiological parameter of the first individual while the physiological parameter of the second individual is being displayed on the display; and redisplaying, on the display, the physiological parameter of the first individual; whereby the physiological parameter of only one individual is displayed at a time.

Alternatively, the method may comprise the steps of displaying the respective physiological parameters of the first individual and displaying the physiological parameter of the second individual; wherein the physiological parameters of each of the first and second individuals are at least simultaneously or alternatively displayed on the display of the electronic device.

It is also to be understood that the following claims are intended to cover all of the generic and specific features of the invention described herein and all statements of the scope of the invention which as a matter of language might fall therebetween.

What is claimed is:

1. A method of displaying, on a display of an electronic device, a physiological parameter of at least a first individual having associated therewith a first biomedical sensing device and a second individual having associated therewith a second biomedical sensing device, the method comprising the steps of:
initiating a communication link between the first biomedical sensing device and the electronic device such that information packets related to the physiological parameter of the first individual are transmitted from the first biomedical sensing device and received and processed by the electronic device;
displaying, on the display, the physiological parameter of the first individual based on the information packets received and processed by the electronic device; and
while maintaining a wireless communication link between the first biomedical sensing device and the electronic device, the method further comprises the steps of:
initiating a wireless communication link between the second biomedical sensing device and the electronic device such that information packets related to the physiological parameter of the second individual are transmitted from the second biomedical sensing device and received and processed by the electronic device;
displaying, on the display, the physiological parameter of the second individual based on the information packets received and processed by the electronic device.

2. The method as claimed in claim 1, including the steps of:
inhibiting the display of the physiological parameter of the first individual while the physiological parameter of the second individual is being displayed on the display; and
redisplaying, on the display, the physiological parameter of the first individual;
whereby the physiological parameter of only one individual is displayed at a time.

3. The method as claimed in claim 1, including the steps of:
displaying the respective physiological parameters of the first individual and displaying the physiological parameter of the second individual;
wherein the physiological parameters of each of the first and second individuals are at least simultaneously or alternatively displayed on the display of the electronic device.

4. The method as claimed in claim 1, wherein the step of initiating a communication link between the second biomedical sensing device and the electronic device comprises the steps of:
selecting the second biomedical sensing device from a plurality of biomedical sensing devices.

5. An arrangement for displaying, on a display of an electronic device, a physiological parameter of at least a first individual having associated therewith a first biomedical sensing device and a second individual having associated therewith a second biomedical sensing device, the arrangement comprising:
means for initiating a communication link between the first biomedical sensing device and the electronic device such that information packets related to the physiological parameter of the first individual are transmitted from the first biomedical sensing device and received and processed by the electronic device;
a display on the electronic device for displaying the physiological parameter of the first individual based on the information packets received and processed by the electronic device; and
means for initiating a wireless communication link between the second biomedical sensing device and the electronic device while maintaining a wireless communication link between the first biomedical sensing device and the electronic device, such that information packets related to the physiological parameter of the second individual are transmitted from the second biomedical sensing device and received and processed by the electronic device;
wherein the electronic device displays the physiological parameter of the second individual based on the information packets received and processed by the electronic device.

6. The arrangement as claimed in claim 5, comprising:
means for inhibiting the display of the physiological parameter of the first individual while the physiological parameter of the second individual is being displayed on the display; and for redisplaying, on the display, the physiological parameter of the first individual;
whereby the physiological parameter of only one individual is displayed at a time.

7. The arrangement as claimed in claim 5, comprising:
means for displaying the respective physiological parameters of the first individual and displaying the physiological parameter of the second individual, wherein the physiological parameters of each of the first and second individuals are at least simultaneously or alternatively displayed on the display of the electronic device.

8. The arrangement as claimed in claim 5, wherein the means for initiating a communication link between the second biomedical sensing device and the electronic device comprises selection means for selecting the second biomedical sensing device from a plurality of biomedical sensing devices.

9. An electronic device that displays a physiological parameter of at least a first individual having associated therewith a first biomedical sensing device and a second individual having associated therewith a second biomedical sensing device, the electronic device comprising:
means for initiating a communication link with the first biomedical sensing device such that information packets related to the physiological parameter of the first individual are transmitted from the first biomedical sensing device and received and processed by the electronic device;
a display for displaying the physiological parameter of the first individual based on the information packets received and processed by the electronic device; and
means for initiating a wireless communication link between the second biomedical sensing device while maintaining a wireless communication link with the first biomedical sensing device, such that information packets related to the physiological parameter of the second individual are transmitted from the second biomedical sensing device and received and processed by the electronic device;
wherein the electronic device displays the physiological parameter of the second individual based on the information packets received and processed by the electronic device.

10. The electronic device as claimed in claim 9, comprising:
means for inhibiting the display of the physiological parameter of the first individual while the physiological parameter of the second individual is being displayed on the display; and for redisplaying, on the display, the physiological parameter of the first individual;
whereby the physiological parameter of only one individual is displayed at a time.

11. The electronic device as claimed in claim 9, comprising:
means for displaying the respective physiological parameters of the first individual and displaying the physiological parameter of the second individual, wherein the physiological parameters of each of the first and second individuals are at least simultaneously or alternatively displayed on the display.

12. The electronic device as claimed in claim 9, wherein the means for initiating a communication link with the second biomedical sensing device comprises selection means for selecting the second biomedical sensing device from a plurality of biomedical sensing devices.
